(19) United States
(12) Patent Application Publication
(10) Pub. No.: US 2024/0238049 A1
(43) Pub. Date: Jul. 18, 2024

(54) MEDICAL INSTRUMENT GUIDANCE SYSTEMS AND ASSOCIATED METHODS

(71) Applicant: INTUITIVE SURGICAL OPERATIONS, INC., Sunnyvale, CA (US)

(72) Inventors: Serena H. Wong, Los Altos, CA (US); Troy K. Adebar, San Jose, CA (US); Mimi Trinh Fitterer, Belmont, CA (US)

(21) Appl. No.: 18/560,314

(22) PCT Filed: May 10, 2022
(86) PCT No.: PCT/US2022/028439

§ 371 Date: Nov. 10, 2023

Related U.S. Application Data
(60) Provisional application No. 63/187,245, filed on May 11, 2021.

(51) Int. Cl. A61B 34/20; A61B 34/10

(52) U.S. Cl. CPC A61B 34/20; A61B 34/10; A61B 2034/105; A61B 2034/2051; A61B 2034/2061; A61B 2034/2065

(57) ABSTRACT

Systems for performing a medical procedure and associated methods and devices are disclosed herein. In some embodiments, a system for performing a medical procedure includes an elongate flexible device configured to be introduced into an anatomic cavity of a patient, and a sensor system carried by the elongate flexible device and configured to obtain location data of the anatomic structure. The system can be configured to perform operations comprising: generating a 3D anatomic model based on first location data, the 3D anatomic model including an object within the anatomic cavity, based on second location data of the elongate flexible device when the object is within the image, and providing guidance for deploying an access tool along an access path through a patient's skin to the object based, at least in part, on the 3D anatomic model.

### CROSS-REFERENCED APPLICATIONS

This application claims priority to and the benefit of U.S. Provisional Application No. 63/187,245, filed May 11, 2021 and entitled “Medical Instrument Guidance Systems and Associated Methods,” which is incorporated by reference herein in its entirety.

### TECHNICAL FIELD

The present disclosure is directed to systems and associated methods for providing guidance for medical procedures.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Some minimally invasive medical tools may be teleoperated or otherwise computer-assisted or delivered by a teleoperated, robotic, or otherwise computer-assisted system. Various features may improve the effectiveness of minimally invasive medical tools and techniques.

### SUMMARY

Embodiments of the present technology are best summarized by the claims that follow the description.

In some embodiments, a system for performing a medical procedure comprises an elongate flexible device configured to be introduced into an anatomic structure of a patient. The system further includes an imaging device coupled to a distal end portion of the elongate flexible device. The imaging device is configured to obtain an image viewed from the distal end portion of the elongate flexible device. The system can also include a sensor system carried by the elongate flexible device and configured to obtain location data of the anatomic structure of the elongate flexible device. The system can further include a processor operably coupled to the elongate flexible device and the sensor system, and a memory operably coupled to the processor. The memory can store instructions that, when executed by the processor, cause the system to perform various operations. The operations can include generating a three-dimensional (3D) anatomic model based, at least in part, on first location data from the sensor system as the elongate flexible device is navigated within an interior space of an anatomic cavity. The 3D anatomic model can include at least one object within the anatomic cavity based on second location data of the elongate flexible device when the object is within the image. The operations can further include providing guidance for deploying an access tool along an access path through the patient's skin to the object based, at least in part, on the 3D anatomic model.

In these and other embodiments, a method for performing a medical procedure includes surveying an anatomic cavity of a patient using an elongate flexible device. The surveying includes receiving commands for navigating the elongate flexible device within an interior space of the anatomic cavity and saving first location data from a first localization sensor coupled to the elongate flexible device. The method also includes receiving first location data from a localization sensor coupled to an elongate flexible device navigated within an anatomic cavity of the patient. The method can continue with generating a 3D anatomic model based, at least in part, on the first location data. The method can include receiving image data from an imaging device coupled to the elongate flexible device and receiving second location data from the localization sensor when at an object within the anatomic cavity is visible in the image data. The method can also include updating the 3D anatomic model to include the object based, at least in part, on the second location data. The method can further include providing guidance for deploying an access tool from an external location to the object in the 3D anatomic model.

In these and further embodiments, a non-transitory, computer-readable medium is provided. The non-transitory, computer-readable instructions stores instructions thereon that, when executed by one or more processors of a computing system, cause the computing system to perform the method of any of the embodiments described herein.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted but are for explanation and understanding only.

FIG. 1 is a flow diagram illustrating a method for performing a medical procedure in accordance with various embodiments of the present technology.

FIG. 2 is a partially schematic illustration of an anatomic structure and an elongate flexible device within the anatomic structure, in accordance with embodiments of the present technology.

FIG. 3 is a flow diagram illustrating a method for generating a 3D anatomic model in accordance with embodiments of the present technology.

FIG. 4 illustrates a representative example of point cloud data generated in accordance with embodiments of the present technology.

FIGS. 5A-5E illustrate various examples of graphical user interfaces for providing guidance for deploying an access tool, in accordance with embodiments of the present technology.

FIG. 6 is a simplified diagram of a teleoperated medical system configured in accordance with various embodiments of the present technology.

FIG. 7A is a simplified diagram of a medical instrument system configured in accordance with various embodiments of the present technology.

FIG. 7B is a simplified diagram of a medical instrument system configured in accordance with various embodiments of the present technology.

### DETAILED DESCRIPTION

The present disclosure is directed to minimally invasive devices and systems for performing medical procedures and associated methods. In some embodiments, a medical procedure includes introducing an elongate flexible device (e.g., a flexible catheter) into an anatomic structure of a patient (e.g., a kidney). The elongate flexible device can include at least one sensor configured to locate at least one target in the anatomic structure (e.g., a kidney stone). Once the target location has been identified, an access tool (e.g., a needle) can be used to create an access path to the target. The access path can be a percutaneous access path for introducing a medical instrument from an external location external to the target. In some embodiments, for example, the medical instrument can be a tool for breaking up a kidney stone in a percutaneous nephrolithotomy (PCNL) procedure, such as a suction tube, nephroscope, or lithotripter.

In such medical procedures, it may be challenging for the operator to position the access tool to create a path to the target while avoiding non-target organs and/or sensitive tissue structures. For example, in a PCNL procedure, the operator may need to create a percutaneous access path to the kidney stone without puncturing the liver, intestines (e.g., bowel, colon, etc.), lungs, and/or nearby blood vessels. However, conventional techniques may not provide sufficient guidance for positioning the access tool. For example, preoperative imaging and/or modeling may be of limited value because the position of the kidney stone, kidney, and/or other organs may shift, e.g., due to differences in the patient's body position during preoperative imaging versus the actual PCNL procedure. Additionally, the kidney and/or surrounding organs can be soft, deformable structures that may change in shape and/or size after preoperative imaging. Additionally, kidney stones may not be visible in certain imaging modalities (e.g., fluoroscopy, computed tomography (CT)). Thus, conventional procedures may rely upon highly trained specialists to perform the initial puncture with the access tool and/or may frequently require multiple attempts to create an access path that is sufficiently on target.

To overcome these and other challenges, the systems and associated methods described herein can be configured to guide an operator in creating an access path to an anatomic target while avoiding nearby sensitive tissue structures. In some embodiments, for example, the system uses an elongate flexible device deployed within the anatomic structure to generate an intraoperative 3D anatomic model of the anatomic structure. The elongate flexible device can include a sensor system configured to obtain sensor data (e.g., location data, point cloud data, image data), and the system can use the sensor data to determine a general 3D shape of the anatomic structure and identify the target location within the 3D shape. Optionally, the elongate flexible device itself (e.g., a portion of the elongate flexible device near the anatomic target) can serve as the target location, which can be advantageous in situations where the anatomy exhibits significant motion (e.g., due to breathing, peristalsis, etc.) since the elongate flexible device can move along with the anatomy. The 3D anatomic model can also include locations of sensitive tissue structures to be avoided (e.g., determined based on generic anatomic models, preoperative data, intraoperative data, operator and/or physician input, etc.). In some embodiments, the system uses the 3D anatomic model to determine an access path for an access tool to reach the target, without passing through the sensitive tissue structures. The system can output a graphical user interface that provides live, accurate guidance for positioning the access tool (e.g., insertion location and/or insertion angle) to create the access path. The 3D anatomic model and/or operator guidance can be updated in real time to reflect any changes in the patient anatomy and/or target that occur during the procedure (e.g., if the kidney stone moves). Accordingly, the approaches disclosed herein can reduce procedure time and complexity, and also improve patient safety by mitigating the risk of injury to non-target organs.

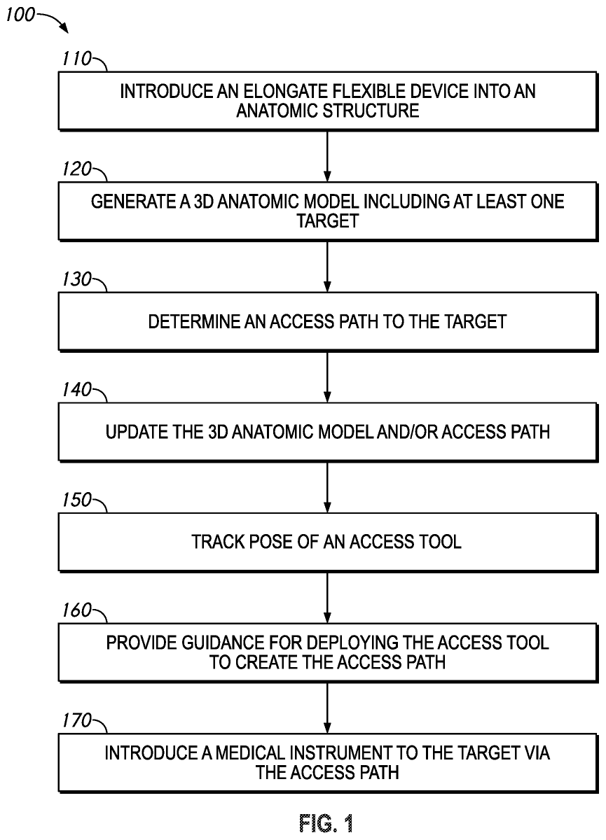

Specific details of several embodiments of the present technology are described herein with reference to FIGS. 1-7B. Although many of the embodiments are described below in the context of navigating and performing medical procedures within a kidney and/or urinary tract of a patient, other applications and other embodiments in addition to those described herein are within the scope of the present technology. For example, unless otherwise specified or made clear from context, the devices, systems, and methods of the present technology can be used for navigating and performing medical procedures on, in, or adjacent other patient anatomy, such as the lungs, gastrointestinal (GI) system, and/or heart of a patient.

It should be noted that other embodiments in addition to those disclosed herein are within the scope of the present technology. For example, although certain embodiments herein are discussed with reference to instruments for accessing and/or breaking up kidney stones, this is not intended to be limiting, and the present technology can also be applied to other types of medical instruments, such as instruments used for diagnosis, treatment, or other medical procedures. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

As used herein, the term “operator” shall be understood to include any type of personnel who may be performing or assisting a procedure and, thus, is inclusive of a physician, a surgeon, a doctor, a nurse, a medical technician, a clinician, other personnel or user of the technology disclosed herein, and any combination thereof. As used herein, the term “patient” should be considered to include human and/or non-human (e.g., animal) patients upon which a medical procedure is being performed.

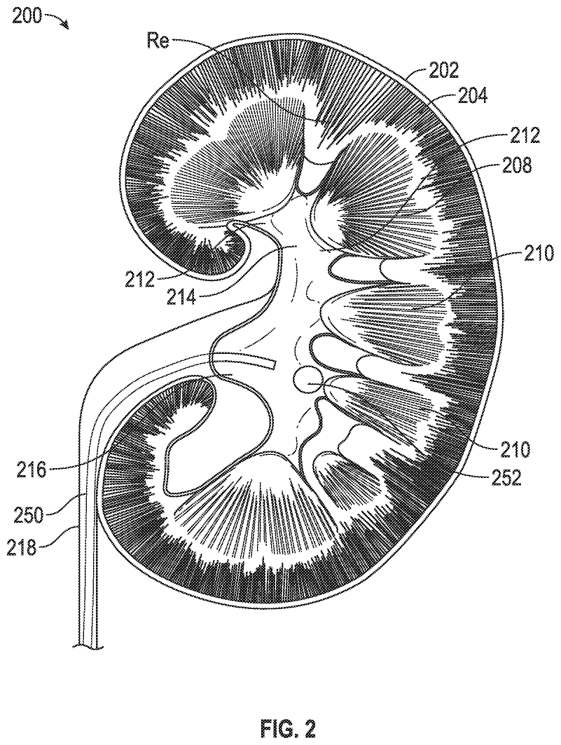

FIG. 1 is a flow diagram illustrating a method 100 for performing a medical procedure in accordance with various embodiments of the present technology. The method 100 is illustrated as a set of steps or processes 110-170. All or a subset of the steps of the method 100 can by implemented by any suitable computing system or device, such as a control system of a medical instrument system or device (e.g., including various components or devices of a robotically-controlled or teleoperated surgical system), a workstation, a portable computing system (e.g., a laptop computer), any/or a combination thereof. In some embodiments, for example, the computing system for implementing the method 100 includes one or more processors operably coupled to a memory storing instructions that, when executed, cause the computing system to perform operations in accordance with the steps 110-170. Additionally or alternatively, all or a subset of the steps 110-170 of the method 100 can be executed at least in part by an operator (e.g., a physician, a user, etc.) of the computing system, and/or by a robotically-controlled surgical system via user inputs from the operator through a user input device or automatically through using closed loop control and/or pre-programmed instructions through a processor of the system. The method 100 is illustrated in the following description by cross-referencing various aspects of FIGS. 2-7B.

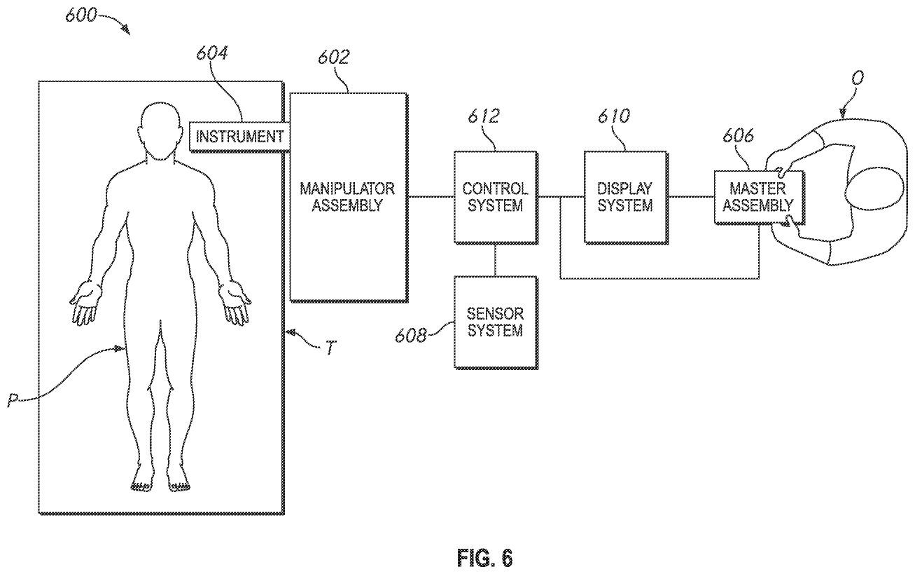

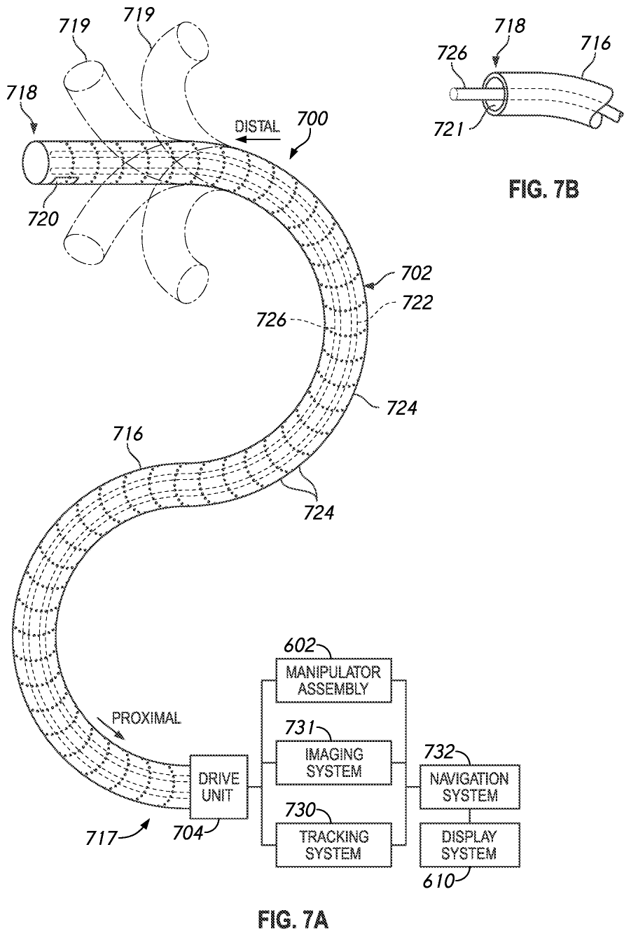

The method 100 begins at step 110 with introducing an elongate flexible device into an anatomic structure of a patient. The elongate flexible device can be a flexible catheter or similar tool suitable for introduction into the anatomic structure via minimally invasive techniques (e.g., via an endoluminal access route). Positioning and/or navigation of the elongate flexible device may be performed manually, the elongate flexible device may be robotically controlled by an operator via an input device, and/or the elongate flexible device may be robotically controlled automatically using a pre-programmed set of instructions from a robotic system. Additional details of elongate flexible devices and robotic medical systems suitable for use with the method 100 are provided below with reference to FIGS. 6-7B.

FIG. 2, for example, is a partially schematic illustration of an anatomic structure 200 and an elongate flexible device 250 within the anatomic structure 200 in accordance with embodiments of the present technology. In the illustrated embodiment, the anatomic structure 200 is a patient's kidney 202. The kidney 202 includes a renal capsule 204, a renal cortex 206, and a renal medulla 208. The renal medulla 208 includes a plurality of renal pyramids 210 containing the nephron structures responsible for urine production. The urine is collected by a series of chambers known as calyces (e.g., minor calyces 212 and major calyces 214). The minor calyces 212 are adjacent to the renal pyramids 210 and converge to form major calyces 214. The major calyces 214 empty into the renal pelvis 216 and ureter 218. The elongate flexible device 250 can be a catheter, ureteroscope, or similar instrument suitable for introduction into the kidney 202 via the patient's urinary tract (e.g., the ureter 218). The elongate flexible device 250 can navigate and/or articulate within the interior spaces of the kidney 202 to reach a target, e.g., a kidney stone 252. The kidney stone 252 may be located near or within the minor calyces 212, major calyces 214, renal pelvis 216, or ureter 218.

Referring again to FIG. 1, at step 120, the method 100 continues with generating a three-dimensional (“3D”) model of the anatomic structure (also referred to herein as a “3D anatomic model”). The 3D anatomic model can be any suitable 3D representation of the passageways, spaces, and/or other features of the anatomic structure, such as a surface model (e.g., a mesh model or other representation of anatomic surfaces, a skeletal model (e.g., a model representing passageways and/or connectivity), or a parametric model (e.g., a model fitting common parameters). The 3D anatomic model can include at least one target, which can be a tissue, object, or any other suitable site to be accessed and/or treated during the medical procedure. For example, in embodiments where the anatomic structure is a kidney, the 3D anatomic model can include the major calyces, minor calyces, renal pelvis, and/or ureter, and the target can be a kidney stone within the kidney, as described in FIG. 2 above. In other embodiments, however, the 3D anatomic model can include other types of anatomic structures and/or targets.

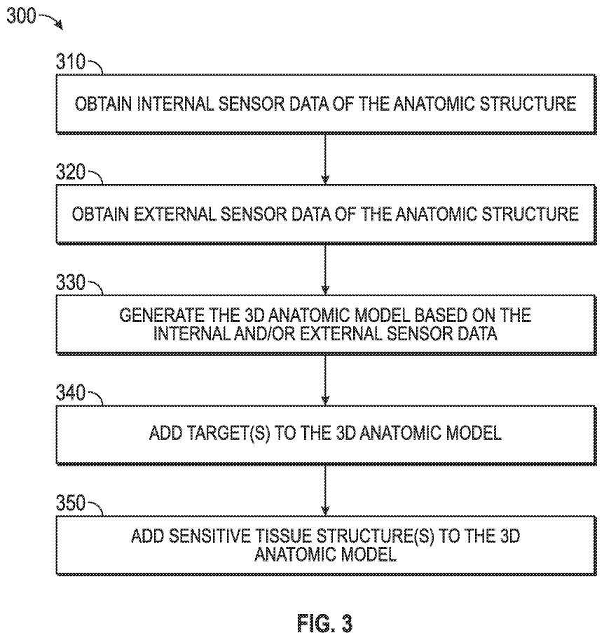

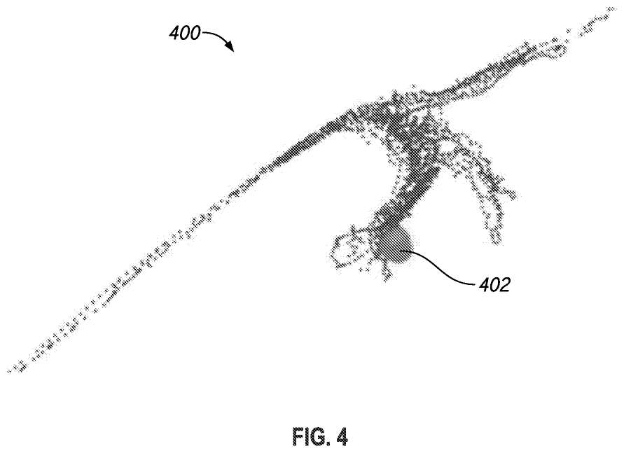

In some embodiments, the 3D anatomic model is generated partially or entirely from intraoperative data obtained during the medical procedure (e.g., while the elongate flexible device is positioned within the anatomic structure). The intraoperative data can include location data (e.g., point cloud data) generated continuously by a localization sensor coupled to the elongate device as the elongate flexible device moves within the anatomic structure. The process of navigating the elongate flexible device within the anatomic structure while obtaining and saving location data generated by the localization sensor may also be referred to herein as “surveying” the anatomic structure, and the location data generated during the surveying process may be referred to herein as “survey location data.” As previously described, location data and/or other intraoperative data may provide a more accurate representation of the current state of the patient anatomy and/or target, compared to preoperative data (e.g., preoperative CT, X-ray, MRI images and/or models) which may be captured a long period of time before performing the medical procedure and/or while a patient is positioned differently than during the medical procedure. A representative method for generating the 3D anatomic model from intraoperative data that can be performed as part of step 120 is described in detail below with reference to FIGS. 3 and 4.

FIG. 3 is a flow diagram illustrating a method 300 for generating a 3D anatomic model in accordance with embodiments of the present technology. The method 300 begins at step 310 with obtaining internal sensor data of an anatomic structure (e.g., an anatomic cavity, such as the interior spaces of a kidney or other organ). The internal sensor data can include, for example, sensor data generated by a sensor system carried by the elongate flexible device. For example, the sensor system can be, or can include, at least one localization sensor configured to generate survey location data as the elongate flexible device surveys the anatomy by driving to various locations within the anatomic structure. The survey location data can be saved to create a cloud of points forming a general shape of the anatomic structure. Any suitable localization sensor can be used, such as a shape sensor, EM sensor, positional sensor, pose sensor, or a combination thereof.

FIG. 4 illustrates a representative example of a point cloud data set 400 generated in accordance with embodiments of the present technology. The point cloud data set 400 can be generated by navigating the elongate flexible device to different locations within the anatomic structure and can provide a 3D representation of the interior spaces and/or passageways of the anatomic structure. In the illustrated embodiment, for example, the point cloud data set 400 depicts the 3D shape of a ureter, renal pelvis, major calyces, and minor calyces of a patient's kidney. The point cloud data set 400 also includes a set of data points corresponding to the location of a target 402 (e.g., a kidney stone) within the anatomic structure. Optionally, the point cloud data set 400 can include data of additional locations within or near the anatomic structure to provide an accurate representation of the relative shape of the anatomy and the location of the target. The point cloud data set 400 can be used to generate a 3D anatomic model of the kidney and kidney stone, as disclosed herein.

Referring again to step 310 of FIG. 3, in some embodiments, the internal sensor data includes other types of data in addition to location data. For example, the internal sensor data can include image data generated by an imaging device within the anatomic structures (e.g., carried by the elongate flexible device). The image data can include, for example, still or video images, ultrasound data, thermal image data, and the like. In some embodiments, each image captured by the imaging device is associated with location data generated by the localization sensor, such the location of an object within the anatomic structure can be determined based on images of the object and the location data associated with the images.

At optional step 320, the method 300 can include obtaining external sensor data of the anatomic structure. The external sensor data can include any data generated by a sensor system external to the patient's body, such as external imaging data generated by an external imaging system. The external image data can include any of the following: CT data, magnetic resonance imaging (MRI) data, fluoroscopy data, thermography data, ultrasound data, optical coherence tomography (OCT) data, thermal image data, impedance data, laser image data, nanotube X-ray image data, and/or other suitable data representing the patient anatomy. The image data can correspond to two-dimensional (2D), 3D, or four-dimensional (e.g., time-based or velocity-based information) images. In some embodiments, for example, the image data includes 2D images from multiple perspectives that can be combined into pseudo-3D images. The external sensor data can include preoperative data and/or intraoperative data.

At step 330, the method 300 continues with generating the 3D anatomic model based on the internal and/or external sensor data. For example, the 3D anatomic model can be generated from the survey location data (e.g., point cloud data using techniques for producing a surface or mesh model from a plurality of 3D data points, such as a surface reconstruction algorithm. In such embodiments, because the sensor system used to generate the point cloud data is carried by the elongate flexible device, the resulting 3D anatomic model may already be in the same reference frame as the elongate flexible device, such that no additional registration step is needed. As another example, a 3D representation can be generated from preoperative image data (e.g., using image segmentation processes), and subsequently combined with the point cloud data to produce the 3D anatomic model. In such embodiments, the method 300 can further include determining a registration between the image data and the point cloud data, e.g., using a registration algorithm such as a point-based iterative closest point (ICP) technique, as described in U.S. Provisional Pat. App. Nos. 62/205,440 and No. 62/205,433, which are both incorporated by reference herein in their entireties.

Optionally, the 3D anatomic model can be generated from both intraoperative data (e.g., internal sensor data, such as location data) and preoperative data (e.g., external image data obtained before the elongate flexible device is introduced into the patient's body). In such embodiments, the intraoperative data can be used to update the preoperative data to ensure that the resulting model accurately represents the current state of the patient anatomy. For example, a preoperative anatomic model can be generated from image data (e.g., CT data) and/or other patient data obtained before the medical procedure, e.g., using image segmentation processes known to those of skill in the art. Subsequently, the preoperative anatomic model can be registered to the intraoperative data (e.g., point cloud data) to place them both in the same reference frame. The registration process can include navigating and/or touching the elongate flexible device to locations of the patient anatomy (e.g., within the anatomic structure) corresponding to known points in the preoperative anatomic model. Alternatively or in combination, the intraoperative data can be registered to the preoperative anatomic model using a registration algorithm (e.g., a point-based ICP technique). Once registered, the intraoperative data can be used to modify the preoperative anatomic model, e.g., by filling in missing portions, resolving errors or ambiguities, etc. If there are portions of the preoperative model that do not match the intraoperative data, the intraoperative data can be assumed to be more accurate and can be used to replace those portions of the preoperative model.

At step 340, the method 300 continues with adding at least one target to the 3D anatomic model. As previously discussed, the target can be an object (e.g., a kidney stone), a tissue to be treated (e.g., biopsied, ablated, etc.), or any other suitable site within the anatomic structure. In some embodiments, step 340 includes generating a model component representing the target and adding that component to the 3D anatomic model. Alternatively or in combination, step 340 can include marking an existing component and/or location in the 3D anatomic model that corresponds to the location of the target in the anatomic structure.

In some embodiments, in order to add the target to the appropriate location in the 3D anatomic model, step 340 further includes identifying the location of the target within the anatomic structure. The target location can be identified, for example, based on internal sensor data generated by a sensor system carried by the elongate flexible device. For example, the sensor system can include an imaging device (e.g., a camera, ultrasound, OCT, etc.) configured to obtain image data of the target. In such embodiments, the elongate flexible device can be navigated within the anatomic structure until the target is within the field of view of the imaging device and is at least partially visible within the image data. The process of imaging and identifying the target can be performed automatically, can be performed based on user input, or suitable combinations thereof. For example, an operator can view the image data (e.g., via a graphical user interface shown on a monitor) and can provide commands via an input device (e.g., touchscreen, mouse, keyboard, joystick, trackball, button, etc.) to indicate the presence of the target in the image data (e.g., by clicking, selecting, marking, etc.). As another example, the operator can drive the elongate flexible device until the target is at a particular location in the image data (e.g., aligned with a visual guide such as a set of crosshairs, centered in the image data, etc.). In yet another example, the method 300 can include analyzing the image data using computer vision and/or machine learning techniques to automatically or semi-automatically identify the target.

Once the target is visible in the image data, step 340 can further include obtaining target location data using a localization sensor (e.g., a shape sensor or EM sensor), and determining the location of the target with respect to the 3D anatomic model based on the target location data and the image data. The target location data obtained in step 340 can be different from the survey location data used to generate in the 3D anatomic model in steps 310 and 330 or can include some or all of the same data points as the target location data. Similarly, the localization sensor can be the same sensor used to obtain the survey location data in step 310 or can be a different sensor. The target location data can indicate the pose of the elongate flexible device while the target is within the field of view of the imaging device. Thus, the target location data can be used to calculate the spatial relationship between the target and the elongate flexible device, which in turn can be used to determine the location of the target in the 3D anatomic model. In embodiments where two different localization sensors are used to generate the survey location data and the target location sensor data, if the relative positions of the two localization sensors are known (e.g., the sensors are both coupled to the elongate flexible device), the target location data can be registered to the survey location data so the target can be positioned appropriately within the 3D anatomic model.

In some embodiments, step 340 of the method 300 also includes determining the distance between the target and the elongate flexible device (or a portion thereof, such as the distal end portion). The distance can be determined in many different ways. For example, the distance can be measured using a proximity sensor (e.g., an optical sensor, time-of-flight sensor, etc.) carried by the elongate flexible device. Alternatively or in combination, the distance can be determined based on the known or estimated geometry (e.g., diameter, height, width) of the target. In such embodiments, the target geometry can be determined or estimated based on image data (e.g., preoperative images) or any other suitable data. Subsequently, the target geometry can be compared to the geometry of the target in the image data to determine the distance between the target and the imaging device (and thus, the elongate flexible device carrying the imaging device). Based on the determined distance, the target can be added to 3D anatomic model at the appropriate location.

Alternatively or in combination, step 340 of the method 300 can include using force, pressure, and/or contact sensor(s) carried by the elongate flexible device to detect the target. This approach can be used in situations where the target has different mechanical properties than the surrounding tissue, such as a different hardness and/or stiffness. In such embodiments, the elongate flexible device can be navigated within the anatomic structure until the force and/or contact sensor detects that the elongate flexible device is in contact with the target. The location of the elongate flexible device (or a portion thereof, such as the distal end portion) at the time of contact can be used as the location of the target.

At step 350, the method 300 can optionally include adding one or more sensitive tissue structures to the 3D anatomic model. The sensitive tissue structures can include any tissue, organ, or other site to be avoided during the medical procedure, e.g., due to risk of injury, side effects, and/or other complications. The sensitive tissue structures can be located nearby but outside of the anatomic structure to be treated. For example, sensitive tissue structures in the context of a kidney-related procedure (e.g., a PCNL procedure) can include the patient's liver, intestines, lungs, and/or blood vessels. In some embodiments, step 350 includes generating one or more model components representing the geometry and/or locations of the sensitive tissue structures and adding the model components to the 3D anatomic model. Alternatively or in combination, step 350 can include marking or otherwise identifying existing components or locations within the 3D anatomic model as corresponding to the locations of the sensitive tissue structures.

In some embodiments, in order to add the sensitive tissue structures to the appropriate locations in the 3D anatomic model, step 350 further includes determining the geometry and/or locations of the sensitive tissue structures relative to the anatomic structure. For example, the geometry and/or locations of the sensitive tissue structures can be estimated based on general anatomic information (e.g., the expected geometry and/or locations for a standard patient) and/or characteristics of the particular patient (e.g., age, sex, height, weight). As another example, the geometry and/or locations of the sensitive tissue structures can be determined based on preoperative data (e.g., CT images). In a further example, the locations of the sensitive tissue structures can be estimated based on known spatial relationships, e.g., knowledge of how the elongate flexible device is positioned relative to the anatomic structure, how the insertion stage for the elongate flexible device is positioned relative to the surgical table, how the patient's body is positioned on the table, and where the sensitive tissue structures are generally located in the patient's body. In yet another example, the locations of the sensitive tissue structures can be estimated by obtaining location data of known anatomic reference points with the elongate flexible device. For instance, a localization sensor can track the location of the elongate flexible device as the elongate flexible device is touched to one or more external and/or internal anatomic reference points (e.g., the ribs), and the tracked location can be used to register the anatomic reference points to the 3D anatomic model. The location of the sensitive tissue structures can then be estimated based on known spatial relationships between the sensitive tissue structures and the anatomic reference points.

In still other embodiments, the locations of the sensitive tissue structures can be estimated based on user input from the operator, a physician or other healthcare professional, etc. For example, a physician could estimate the locations of sensitive tissue structures in the patient, e.g., by manually palpating the patient. The physician (or another operator) could then mark these locations by touching the elongate flexible device to the corresponding locations on the patient's external and/or internal anatomy. The marked locations can be used to define a space or region that should be avoided during the procedure. In other embodiments, sensors (e.g. location sensors integrated into a patient patch or other structure) may be coupled to patient anatomy at locations of sensitive tissue. In other embodiments, sensors (e.g. location sensors integrated into a patient patch or other structure) may be coupled to patient anatomy at locations of sensitive tissue.

In some embodiments, the geometry and/or locations of the sensitive tissue structures determined in step 350 are initial estimates, and the 3D anatomic model can subsequently be further updated to refine these estimates, if appropriate. The process for updating the 3D anatomic model is described further below with reference to step 140 of FIG. 1.

Referring again to FIG. 1, the method 100 continues at step 130 with determining an access path to the target, based on the generated 3D anatomic model. The access path can be a planned route for introducing a medical instrument to the target via minimally invasive techniques. For example, the access path can provide a percutaneous route to the target from a location external to the patient's body. The access path can be determined based on various factors, such as path length (e.g., the shortest path to the target), path shape (e.g., a straight path may be appropriate for procedures using rigid instruments, a curved path may be appropriate for procedures using flexible instruments), avoiding intersecting with or passing too close to sensitive tissue structures, and/or optimal approach to a target organ. In some embodiments, step 130 further includes determining an insertion position and/or angle for an access tool (e.g., a needle, cannula, etc.) to create an initial puncture, incision, or other opening for the access path. The insertion position and/or angle can be aligned with (e.g., parallel to) the trajectory of the access path.

Optionally, step 130 can include displaying the determined access path to an operator so the operator can review the access path and provide feedback, if appropriate. For example, step 130 can include presenting a graphical user interface including the access path overlaid onto the 3D anatomic model. The operator can view the access path and provide feedback to accept, reject, or modify the path (e.g., via an input device such as a mouse, keyboard, joystick, touchscreen, etc.). In some embodiments, step 130 includes generating multiple access paths (e.g., multiple entry points/paths, different path lengths, shapes, insertion locations, etc.), and the operator can select a particular path to be used in the procedure based on desirability (e.g., distance to critical structures, path length, etc.).

At step 140, the method 100 optionally includes updating the 3D anatomic model and/or access path, based on intraoperative data (e.g., image data, location data, user input, etc.). Updates to the model may be appropriate, for example, if the target, anatomic structure, and/or sensitive tissue structures move or otherwise change during the procedure. Additionally, the 3D anatomic model can be updated to more accurately conform to the actual geometry and/or locations of the target, anatomic structure, and/or sensitive tissue structures. For example, as previously discussed, the geometry and/or locations of the sensitive tissue structures in the 3D anatomic model can be initial estimates that are subsequently updated once intraoperative data is available. As another example, when the target moves within anatomy, the target location in the 3D anatomic model can be updated, e.g., by moving a distal section of the elongate flexible device to a plurality of different positions to maintain the target within the field of view of a camera coupled to the elongate flexible device. The elongate flexible device (and the camera coupled thereto) may be user controlled (e.g., manually navigated and/or robotically controlled via operator control through an input device) and/or automatically controlled (e.g., using a pre-programmed set of instructions from a robotic system). The access path can also be updated to account for the changes to the 3D anatomic model, if appropriate. The 3D anatomic model and/or access path can be updated at any suitable frequency, such as continuously, periodically at predetermined time intervals (e.g., once every x number of seconds, minutes, etc.), when new sensor data is received, when significant changes are detected (e.g., if the target moves), in response to user input, and/or combinations thereof.

In some embodiments, the 3D anatomic model is updated based on intraoperative image data obtained during the medical procedure, such as CT data, fluoroscopy data, ultrasound data, etc. The image data can be obtained by an external imaging system, by an imaging device within the patient's body (e.g., carried by the elongate flexible device), or a combination thereof. The image data can be analyzed to identify the current geometry and/or locations of the target, anatomic structure, and/or sensitive tissue structures, such as based on user input, using computer vision and/or machine learning techniques, and/or a combination thereof. The current geometry and/or locations of the target, anatomic structure, and/or sensitive tissue structures can be compared to the 3D anatomic model to identify any significant differences (e.g., changes in shape, size, location, etc.). If appropriate, the 3D anatomic model can be revised to reflect the current geometry and/or locations depicted in the image data. Optionally, the revisions can be presented to the operator for feedback (e.g., approval, rejection, or modification) before being incorporated in the model.

Optionally, step 140 can include registering the intraoperative data to the 3D anatomic model so that the geometry and/or locations in the intraoperative data can be mapped onto the model. For example, in embodiments where the intraoperative data includes image data obtained with external imaging systems, the registration process can include obtaining image data of the elongate flexible device or a portion thereof (e.g., the distal end portion) and identifying the elongate flexible device in the image data. The identification can be performed automatically (e.g., using computer vision and/or machine learning techniques), based on user input, or combinations thereof. Optionally, the elongate flexible device can be positioned in a shape to facilitate identification (e.g., a hooked shape). Examples of registration processes based on image data of an elongate flexible device are provided in International Publication No. WO 2017/139621, filed Feb. 10, 2017, disclosing “Systems and Methods for Using Registered Fluoroscopic Images in Image-Guided Surgery,” which is incorporated by reference herein in its entirety. In some embodiments, the registration process of step 140 can alternatively or additionally be performed at a different stage in the method 100, e.g., as part of any of steps 110-130.

At step 150, the method 100 optionally includes tracking a pose of an access tool relative to the 3D anatomic model. As previously discussed, the access tool can be a needle or other suitable medical instrument for creating the access path, and the tracked pose (e.g., position, orientation, location) can be used to guide an operator in deploying the access tool along the access path, as discussed further below. The access tool may be positioned manually, the access tool may be robotically controlled by operator control through an input device, or the access tool may be robotically controlled automatically using a pre-programmed set of instructions from a robotic system (as will be described in further detail below with reference to FIGS. 6-7B).

The pose of the access tool can be tracked in many different ways, such as using a localization sensor (e.g., shape sensor, EM sensor), an imaging device (e.g., ultrasound, fluoroscopy, CT), a support structure having a known spatial and/or kinematic relationship with the access tool (e.g., a mechanical jig, needle guide, insertion stage, etc.), or suitable combinations thereof. For example, the access tool can include a localization sensor configured to generate location data of the access tool. The localization sensor can be configured to be removably coupled to the access tool (e.g., a sensor fiber or other component inserted within a working channel or lumen) or can be permanently affixed to the access tool. Additional examples of techniques for incorporating a localization sensor in an access tool are provided in U.S. Pat. No. 9,636,040, filed Jan. 28, 2013, disclosing “Steerable Flexible Needle with Embedded Shape Sensing,” which is incorporated by reference in its entirety.

In some embodiments, the access tool localization sensor is registered to the flexible device localization so that the pose of the access tool can be tracked relative to the elongate flexible device (and thus, the reference frame of the 3D anatomic model). The registration can be performed in various ways. For example, the first and second localization sensors can be placed in a known spatial relationship with each other during a setup procedure, e.g., manually by the operator and/or using a 3D guide, block, plate, etc., that includes cutouts or other patterning for positioning the sensors in a predetermined configuration. As another example, the first and second localization sensors can be touched to the same set of reference points on the patient's body and/or another object. In a further example, the first and second localization sensors can be coupled to the same support structure such that their relative spatial configuration is known. For instance, the proximal end portions of both sensors can be mounted to the same insertion stage or other structural support. In still another example, the first and second localization sensor can be coupled to different support structures, but the spatial configuration and/or kinematics between the different structures is known and can be used to calculate the spatial relationship between the sensors. For instance, the proximal end portion of the first localization sensor can be mounted to a first insertion stage, robotic arm, etc., while the proximal end portion of the second localization sensor can be mounted to a second insertion stage, robotic arm, etc. As yet another example, the first and second localization sensors can be or include a receiver-transmitter pair, and the signals communicated between the receiver-transmitter pair can be used to determine the spatial relationship between the sensors.

In other embodiments, however, the localization sensor used to track the access tool can be the same localization sensor used to generate the survey location data of the elongate flexible device in step 120. In such embodiments, the localization sensor can be a removable sensor (e.g., a sensor fiber) configured to be sequentially coupled to (e.g., inserted in a working lumen of) the elongated flexible device and the access tool. The localization sensor can first be coupled to the elongate flexible device to obtain data of the anatomic structure and target, as previously discussed with respect to step 120. In some embodiments, once the target is detected (e.g., based on user input, image data, etc., as described above), the elongate flexible device is oriented toward the target and the localization sensor is used to record the pose of the elongate flexible device. The recorded pose can be used to determine the location of the target with respect to the elongate flexible device and/or 3D anatomic model, as described above. Subsequently, the localization sensor can be withdrawn from the elongate flexible device and coupled to the access tool to track the pose of the access tool, in connection with step 150. In some embodiments, because the same localization sensor is used for both the elongate flexible device and the access tool, no registration is needed to map the access tool pose data to the 3D anatomic model.

As another example, the access tool can include an imaging device (e.g., an ultrasound device) configured to generate image data (e.g. 3D Doppler images). The imaging device can be removably coupled to the access tool (e.g., inserted within a working channel or lumen) or can be permanently affixed to the access tool. The image data can be used to generate a 3D representation of the patient anatomy in the reference frame of the access tool. Subsequently, the 3D representation can be registered or otherwise compared to the 3D anatomic model to determine the pose of the access tool relative to the 3D anatomic model and/or update the 3D anatomic model and virtual image of the access tool within the 3D anatomic model.

In a further example, the access tool can be tracked using intraoperative image data (e.g., fluoroscopy, CT) generated by an imaging device separate from the access tool (e.g., an external imaging system). Depending on the particular imaging modality used, the image data can include views of the access tool from multiple imaging planes to facilitate continuous tracking (e.g., for fluoroscopy, multiple 2D views may be needed to track the 3D pose of the access tool). The access tool can be automatically or semi-automatically tracked in the image data based on the known geometry of the access tool, fiducials or other markers on the access tool, user input, etc. Optionally, the access tool can include a localization sensor, and the survey location data generated by the localization sensor can be used as guidance for orienting the imaging device to capture images of the access tool (e.g., for fluoroscopy, the imaging device can be adjusted so the access tool is parallel to the fluoroscopic imaging plane, which may be more suitable for tracking purposes). The intraoperative image data can then be registered to the 3D anatomic model so the pose of the access tool in the image data can be determined relative to the 3D anatomic model (e.g., using the techniques previously described in step 140). Alternatively or in combination, the imaging device can obtain image data of the access tool together with the elongate flexible device so the pose of the access tool can be determined relative to the elongate flexible device (which can be in the same reference frame as the 3D anatomic model).

At step 160, the method 100 can include providing guidance for deploying the access tool to create the access path. The guidance can be presented to the user as a graphical user interface displaying various information, such as a graphical representation of the 3D anatomic model including the anatomic structure, target, and/or nearby sensitive tissue structures. The graphical user interface can also show the access path determined in step 130 (e.g., as a virtual line or similar visual element overlaid onto the 3D anatomic model). Additionally, the graphical user interface can show the locations of various medical instruments with respect to the 3D anatomic model, such as including virtual renderings representing the real time locations of the elongate flexible device and/or access tool. Optionally, the graphical user interface can display the 3D anatomic model from a plurality of different virtual views, such as a global view showing the entire anatomic region, an access tool point of view, and/or an elongate flexible device point of view.

As the operator positions the access tool relative to the patient's body (e.g., manually or via a robotically-controlled system), the graphical user interface can provide instructions, feedback, notifications, alerts, etc., to guide the operator in inserting the access tool into the patient's body along the planned access path. For example, the graphical user interface can display a target insertion location (e.g., an external site on the patient's body) and/or a target insertion angle for the access tool to make the initial puncture for the access path. The graphical user interface can also show the current location and/or angle of the access tool (e.g., based on the tracked pose of the access tool of step 150) relative to the target site, a point of initial puncture, the sensitive tissue structures, and/or the kidney, and, if appropriate, provide feedback (e.g., visual, audible, haptic, etc.) guiding the operator to adjust current location and/or angle of the access tool toward the target location and/or angle, respectively.

Once the initial puncture has been made, the graphical user interface can track the current pose of the access tool with respect to the planned access path, target, and/or local anatomy as the operator inserts the access tool into the patient's body. In some embodiments, the graphical user interface outputs alerts or other feedback (e.g., visual, audible, haptic, etc.) if the access tool deviates from the planned access path, approaches sensitive tissue structures, or otherwise requires correction. The graphical user interface can be updated (e.g., as previously discussed with respect to steps 140 and 150) to provide real-time monitoring and feedback until the access tool reaches the target.

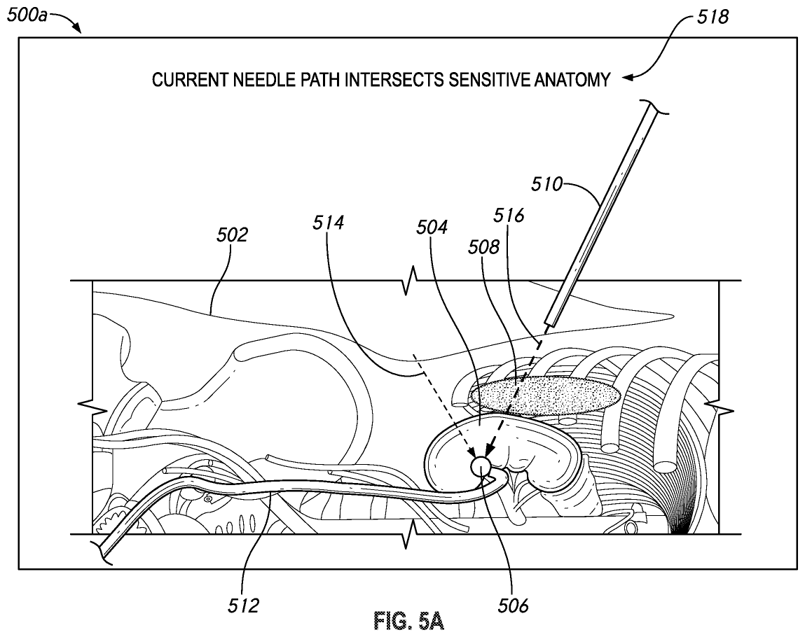

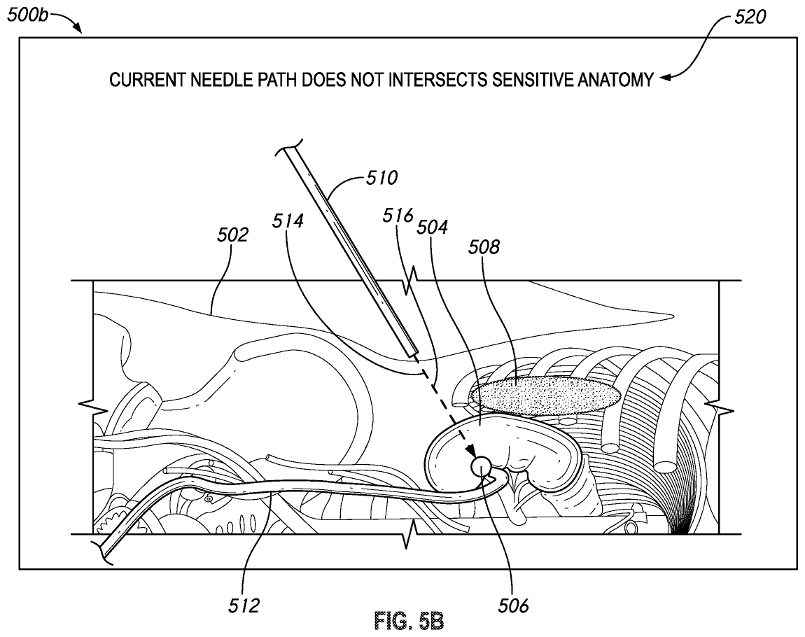

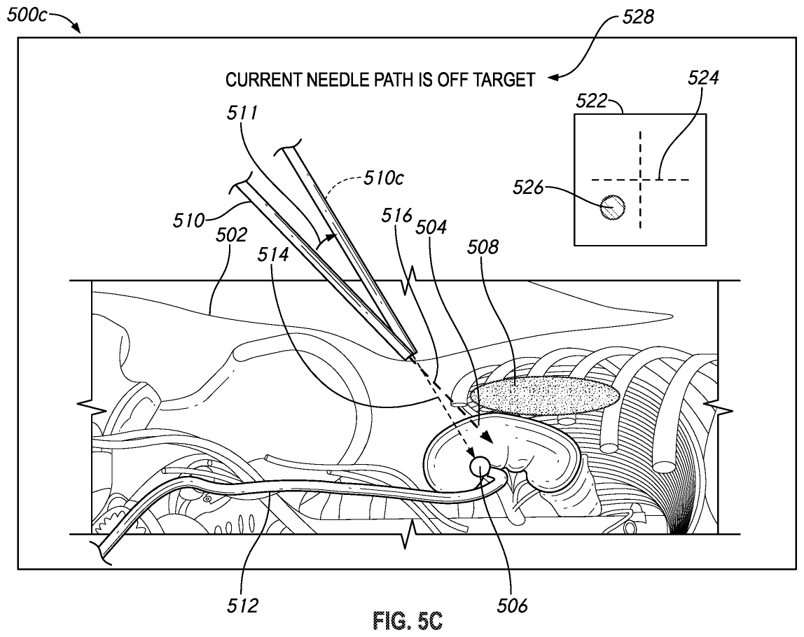

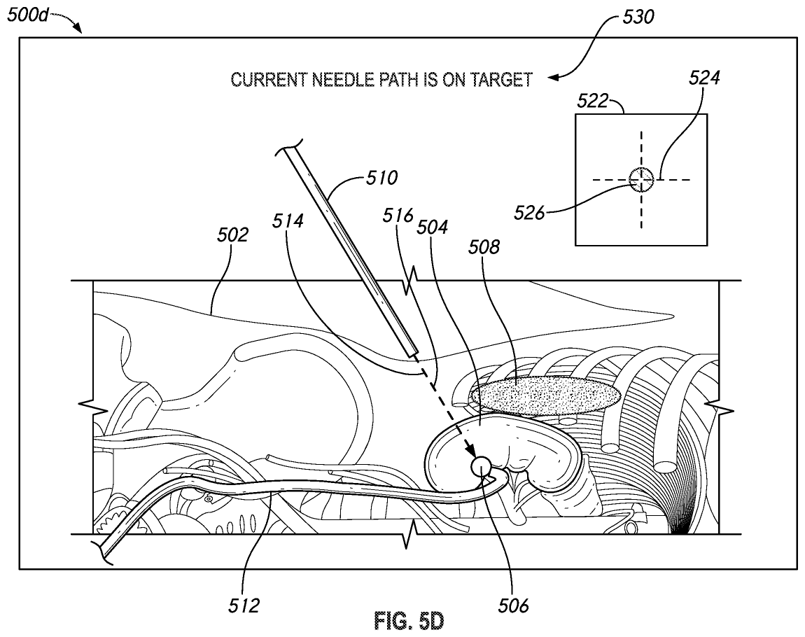

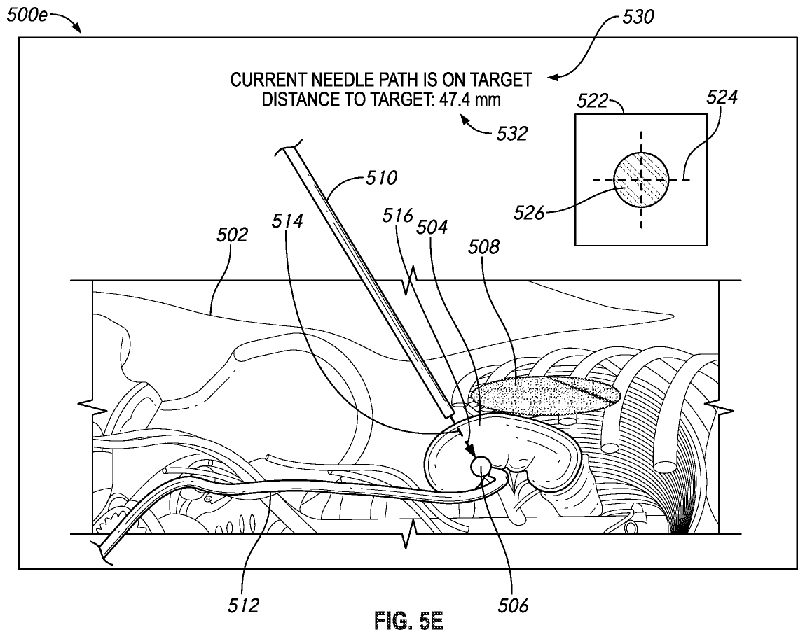

FIGS. 5A-5E are partially schematic illustrations of various examples of graphical user interfaces 500a-500e (“interfaces 500a-500e”) for providing guidance for deploying an access tool in accordance with embodiments of the present technology. The features of the interfaces 500a-500e can be combined with each other and/or with any of the other embodiments described herein. Referring first to FIG. 5A, the interface 500a displays a graphical representation of a 3D anatomic model 502, including the locations of the anatomic structure 504 (e.g., a kidney), target 506 (e.g., a kidney stone), and a sensitive tissue structure 508 near the anatomic structure 504. The interface 500a can also include a representation of the access tool 510 and, optionally, the elongate flexible device 512. In other embodiments, rather than showing the entire access tool 510 and/or elongate flexible device 512, the interface 500a can show only a portion of these devices (e.g., only the distal end portions of the access tool 510 and/or elongate flexible device 512). The interface 500a can also display a planned access path 514 (shown in broken lines) for the access tool 510 to reach the target 506. As shown in FIG. 5A, the planned access path 514 can be a line, vector, or other visual indicia overlaid onto the 3D anatomic model 502. The interface 500a can also show a projected access path 516 (shown in broken lines) for the access tool 510, e.g., the path the access tool 510 would take if introduced into the patient's body at the current insertion location and angle.

In the illustrated embodiment, the projected access path 516 of the access tool 510 intersects the sensitive tissue structure 508. Accordingly, the interface 500a can present feedback (e.g., a message 518 and/or other visual, audible, and/or haptic alerts) informing the operator of this issue. The interface 500a can further instruct the operator to correct the positioning of the access tool 510 (e.g., by adjusting the current insertion location and/or angle) so the projected access path 516 will not pass through or too close to the sensitive tissue structure 508. For example, the interface 500a can instruct the operator to reposition the access tool 510 relative to the patient's body so the projected access path 516 is aligned with the planned access path 514.

Referring next to FIG. 5B, the access tool 510 has been moved relative the patient such that its projected access path 516 is aligned with the planned access path 514 and no longer intersects the sensitive tissue structure 508. Accordingly, the interface 500b can provide feedback to the operator (e.g., a message 520) indicating that current path is satisfactory, and the operator can proceed with inserting the access tool 510 into the patient's body.

Referring next to FIG. 5C, in some embodiments, the interface 500c includes a targeting indicator 522 to guide the operator in aligning the access tool 510 with the target 506. As shown in FIG. 5C, the targeting indicator 522 can include a set of crosshairs 524 representing the projected access path 516 (e.g., viewed from a plane normal to the projected access path 516), and a visual element 526 representing the location of the target 506 relative to the projected access path 516. In the illustrated embodiment, the access tool 510 is currently off target since the projected access path 516 does not intersect the target 506. This is shown in targeting indicator 522 by the visual element 526 being offset from the crosshairs 524. The interface 500c can also provide visual, audible, and/or haptic feedback (e.g., message 528) alerting the operator that the access tool 510 is currently off target. Additionally, the interface 500c can show the planned access path 514, a representation of the access tool 510c in the correct location and insertion angle to create the planned access path 514, and visual indicators (e.g., arrow 511) instructing the operator on how to adjust the current location and/or angle of the access tool 510. As the operator adjustably moves the access tool 510, the various elements of the interface 500c (e.g., projected access path 516, targeting indicator 522) can be updated to provide real time guidance and feedback.

Referring next to FIG. 5D, the access tool 510 has been adjusted so that the projected access path 516 is aligned with the target 506 and the planned access path 514. This can be represented in the interface 500d via the targeting indicator 522, which shows the crosshairs 524 intersecting with the visual element 526. Additionally, the interface 500d can provide feedback (e.g., message 530) indicating that the access tool 510 is on target and is ready to be inserted into the patient's body.

Referring next to FIG. 5E, as the access tool 510 is inserted into the patient's body, the interface 500e can track the distance between the distal end portion of the access tool 510 and the target 506, e.g., via message 532 and/or other visual, audible, or haptic feedback. Optionally, the size of the visual element 526 in the targeting indicator 522 can be adjusted to reflect the distance between the access tool 510 and the target 506, e.g., the visual element 526 is smaller when the access tool 510 is far from the target 506 and is larger when the access tool 510 is close to the target 506. Additionally, the lengths of the planned and projected access paths 514, 516 can be updated in real time to depict the remaining distance between the access tool 510 and the target 506.

Referring again to step 160 of FIG. 1, in some embodiments, the graphical user interface displayed to the operator can include live image data from an imaging device, such as an external imaging system and/or internal imaging device within the patient's body. The imaging device can be the same imaging device used to update the 3D anatomic model (step 140) and/or track the access tool (step 150), or a different imaging device may be utilized. The image data can be presented together with the graphical representation of the 3D anatomic model so the operator can view and compare the actual pose of the access tool with the planned access path. Alternatively or in combination, various components of the 3D anatomic model can be overlaid onto the image data, such as the planned access path, target, sensitive tissue structures, etc.

In some embodiments, the graphical user interface also displays instructions, feedback, notifications, etc., for adjusting the imaging device to capture images of the access tool. This approach can be used in situations where different imaging planes are advantageous for different procedure steps. For example, when making the initial puncture with the access tool, the instructions can direct the operator to use an imaging plane normal or substantially normal to the planned access path so that the access path is shown as a point or small region on the patient's body. A normal imaging plane can help the operator place the distal tip of the access tool at the correct location. Optionally, a laser dot or similar visual indicator can be projected onto the patient's body to mark the insertion location.

Once the initial puncture has been made, the instructions can then direct the operator to use an imaging plane parallel or substantially parallel to the planned access path. A parallel imaging plane can provide a clearer view of the pose of the access tool as it is inserted into the body. In some embodiments, step 160 further includes monitoring the position and/or orientation of the imaging device (or a portion thereof, such as imaging arm) to instruct the operator on how to achieve the correct imaging plane and/or confirm that the correct imaging plane is being used.

Referring again to FIG. 1, at step 170, the method 100 includes introducing a medical instrument to the target via the access path. In some embodiments, once the access tool has reached the target, the access tool is withdrawn so a medical instrument can be introduced to the target via the access path. Alternatively, the access tool can remain in the patient's body, and the medical instrument can be introduced into the patient's body via a working lumen or channel in the access tool. In other embodiments, however, the access tool itself can be used to treat the target, such that step 170 is optional and can be omitted.

The medical instrument can be any minimally invasive instrument or tool suitable for use in, for example, surgical, diagnostic, therapeutic, ablative, and/or biopsy procedures. For example, the medical instrument can be a suction tube, nephroscope, lithotripter, ablation probe, biopsy needle, or other device used to treat the target. The positioning of the medical instrument may be performed manually, the medical instrument may be robotically controlled by operator control through an input device, or the medical instrument may be robotically controlled automatically using a pre-programmed set of instructions from a robotic system (as will be described further below with reference to FIGS. 6-7B).

Optionally, the graphical user interface provided in step 160 can also be used to guide the operator when introducing the medical instrument into the patient's body. For example, the pose of the medical instrument relative to the 3D anatomic model can be tracked (e.g., using the techniques described above in steps 150 and 160). Alternatively or in combination, the graphical user interface can show live image data from a separate imaging device so the operator can visualize the location of the medical instrument within the patient anatomy. The image data can depict the medical instrument from a single imaging plane, or from multiple imaging planes. In some embodiments, for example, the medical instrument is imaged from an imaging plane parallel or substantially parallel to the access path, which may be helpful for visualizing the pose of the medical instrument. Optionally, the medical instrument itself can include an imaging device or other sensor system so the operator can monitor the location of the medical instrument and/or treatment progress from the point of view of the medical instrument.

Although the steps of the method 100 are discussed and illustrated in a particular order, the method 100 illustrated in FIG. 1 is not so limited. In other embodiments, the method 100 can be performed in a different order. In these and other embodiments, any of the steps of the method 100 can be performed before, during, and/or after any of the other steps of the method 100. For example, step 140 can be performed before, during, and/or after any of steps 150, 160, and/or 170; step 150 can be performed before, during, and/or after any of steps 110-140 or 160; and and/or step 160 can be performed before, during, and/or after steps 140 and/or 150. Additionally, one or more steps of the method 100 can be repeated (e.g., any of steps 130-160).

Optionally, one or more steps of the method 100 illustrated in FIG. 1 can be omitted (e.g., steps 140 and/or 150). For example, in embodiments where the access tool is not tracked (i.e., step 150 is omitted), the method 100 can instead include registering the 3D anatomic model to live intraoperative image data (e.g., fluoroscopy data) so that the operator can track the location of the target, anatomic structure, and/or sensitive tissue structures relative to the live images. In such embodiments, the graphical user interface can overlay visual indicators (e.g., highlighting, shading, markings) representing the target, anatomic structure, and/or sensitive tissue structures onto the corresponding components in the live image data. The elongate flexible device and/or access tool can be visible in the live image data so that the operator can assess their locations relative to the patient anatomy.

Moreover, a person of ordinary skill in the relevant art will recognize that the illustrated method 100 can be altered and still remain within these and other embodiments of the present technology. For example, although certain embodiments of the method 100 are described above with reference to a percutaneous access path, in other embodiments, the method 100 can be applied to other types of access paths. For example, the access tool can be introduced via an endoluminal access path, e.g., through a working channel or lumen of the elongate flexible device. In such embodiments, because the pose of the access tool corresponds to the pose of the elongate flexible device, the method 100 can omit determining an access path for the access tool (step 130) and/or tracking the pose of the access tool (step 150). Instead, the guidance provided in step 160 can focus on tracking and updating the location of the target, e.g., in case the target moves during the procedure.

Additionally, in other embodiments, the guidance provided by the method 100 can simply include directing the access tool toward the elongate flexible device (e.g., toward a distal end portion or other portion of the elongate flexible device near the target). In such embodiments, the method 100 does not need to determine a precise access path to the target (i.e., step 130 can be omitted). Instead, the method 100 can simply include tracking the relative locations of the access tool and elongate flexible device, such as by respective localization sensors on the access tool and elongate flexible device, a receiver on the access tool paired with a transmitted on the elongate flexible device (or vice-versa), and/or other suitable techniques. The guidance provided to the operator in step 160 can show the locations of the access tool and elongate flexible device relative to each other and/or to the 3D anatomic model. Optionally, the access tool can include an imaging device (e.g., an ultrasound device) and/or other sensor system to help the operator avoid sensitive tissue structures when inserting the access tool into the patient's body.

FIG. 6 is a simplified diagram of a teleoperated medical system 600 (“medical system 600”) configured in accordance with various embodiments of the present technology. The medical system 600 can be used to perform any of the processes described herein in connection with FIGS. 1-5E. For example, the medical system 600 can be used to perform a medical procedure including mapping an anatomic structure with an elongate flexible device and creating an access path with an access tool, as previously discussed in connection with the method 100 of FIG. 1.

In some embodiments, the medical system 600 may be suitable for use in, for example, surgical, diagnostic, therapeutic, or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

As shown in FIG. 6, the medical system 600 generally includes a manipulator assembly 602 for operating a medical instrument 604 in performing various procedures on a patient P positioned on a table T. In some embodiments, the medical instrument 604 may include, deliver, couple to, and/or control any of the flexible instruments described herein. The manipulator assembly 602 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated.

The medical system 600 further includes a master assembly 606 having one or more control devices for controlling the manipulator assembly 602. The manipulator assembly 602 supports the medical instrument 604 and may optionally include a plurality of actuators or motors that drive inputs on the medical instrument 604 in response to commands from a control system 612. The actuators may optionally include drive systems that when coupled to the medical instrument 604 may advance the medical instrument 604 into a naturally or surgically created anatomic orifice. Other drive systems may move the distal end of the medical instrument 604 in multiple degrees of freedom, which may include three degrees of linear motion (e.g., linear motion along the X, Y, and Z Cartesian axes) and in three degrees of rotational motion (e.g., rotation about the X, Y, and Z Cartesian axes). Additionally, the actuators can be used to actuate an articulable end effector of the medical instrument 604 for grasping tissue in the jaws of a biopsy device and/or the like. Actuator position sensors such as resolvers, encoders, potentiometers, and other mechanisms may provide sensor data to the medical system 600 describing the rotation and orientation of the motor shafts. This position sensor data may be used to determine motion of the objects manipulated by the actuators.

The medical system 600 also includes a display system 610 for displaying an image or representation of the surgical site and the medical instrument 604 generated by sub-systems of a sensor system 608 and/or any auxiliary information related to a procedure including information related to ablation (e.g., temperature, impedance, energy delivery power levels, frequency, current, energy delivery duration, indicators of tissue ablation, etc.). The display system 610 and the master assembly 606 may be oriented so an operator O can control the medical instrument 604 and the master assembly 606 with the perception of telepresence.

In some embodiments, the medical instrument 604 may include components of an imaging system, which may include an imaging scope assembly or imaging instrument that records a concurrent or real-time image of a surgical site and provides the image to the operator O through one or more displays of the medical system 600, such as one or more displays of the display system 610. The concurrent image may be, for example, a two or three-dimensional image captured by an imaging instrument positioned within the surgical site. In some embodiments, the imaging system includes endoscopic imaging instrument components that may be integrally or removably coupled to the medical instrument 604. In some embodiments, however, a separate endoscope, attached to a separate manipulator assembly may be used with the medical instrument 604 to image the surgical site. In some embodiments, the imaging system includes a channel (not shown) that may provide for a delivery of instruments, devices, catheters, and/or the flexible instruments described herein. The imaging system may be implemented as hardware, firmware, software, or a combination thereof which interact with or are otherwise executed by one or more computer processors, which may include the processors of the control system 612.

The medical system 600 may also include the control system 612. The control system 612 includes at least one memory and at least one computer processor (not shown) for effecting control the between medical instrument 604, the master assembly 606, the sensor system 608, and the display system 610. The control system 612 also includes programmed instructions (e.g., a non-transitory machine-readable medium storing the instructions) to implement some or all of the methods described in accordance with aspects disclosed herein, including instructions for providing information to the display system 610.

The control system 612 may optionally further include a virtual visualization system to provide navigation assistance to the operator O when controlling the medical instrument 604 during an image-guided surgical procedure. Virtual navigation using the virtual visualization system may be based upon reference to an acquired preoperative or intraoperative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 7A is a simplified diagram of a medical instrument system 700 configured in accordance with various embodiments of the present technology. The medical instrument system 700 includes an elongate flexible device 702, such as a flexible catheter, coupled to a drive unit 704. The elongate flexible device 702 includes a flexible body 716 having a proximal end 717 and a distal end or tip portion 718. The medical instrument system 700 further includes a tracking system 730 for determining the position, orientation, speed, velocity, pose, and/or shape of the distal end 718 and/or of one or more segments 724 along the flexible body 716 using one or more sensors and/or imaging devices as described in further detail below.

The tracking system 730 may optionally track the distal end 718 and/or one or more of the segments 724 using a shape sensor 722. The shape sensor 722 may optionally include an optical fiber aligned with the flexible body 716 (e.g., provided within an interior channel (not shown) or mounted externally). The optical fiber of the shape sensor 722 forms a fiber optic bend sensor for determining the shape of the flexible body 716. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. No. 7,781,724, filed Sep. 26, 2006, disclosing “Fiber Optic Position and Shape Sensing Device and Method Relating Thereto”; U.S. Pat. No. 7,772,541, filed Mar. 12, 2008, disclosing “Fiber Optic Position and/or Shape Sensing Based on Rayleigh Scatter”; and U.S. Pat. No. 6,389,187, filed Apr. 21, 2000, disclosing “Optical Fiber Bend Sensor,” which are all incorporated by reference herein in their entireties. In some embodiments, the tracking system 730 may optionally and/or additionally track the distal end 718 using a position sensor system 720. The position sensor system 720 may be a component of an EM sensor system with the position sensor system 720 including one or more conductive coils that may be subjected to an externally generated electromagnetic field. In some embodiments, the position sensor system 720 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, filed Aug. 9, 1999, disclosing “Six-Degree of Freedom Tracking System Having a Passive Transponder on the Object Being Tracked,” which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g. fiber shape sensors, EM sensors, and/or the like) may be integrated within the medical instrument 726 and used to track the position, orientation, speed, velocity, pose, and/or shape of a distal end or portion of medical instrument 726 using the tracking system 730.

The flexible body 716 includes a channel 721 sized and shaped to receive a medical instrument 726. FIG. 7B, for example, is a simplified diagram of the flexible body 716 with the medical instrument 726 extended according to some embodiments. In some embodiments, the medical instrument 726 may be used for procedures such as imaging, visualization, surgery, biopsy, ablation, illumination, irrigation, and/or suction. The medical instrument 726 can be deployed through the channel 721 of the flexible body 716 and used at a target location within the anatomy. The medical instrument 726 may include, for example, energy delivery instruments (e.g., an ablation probe), image capture probes, biopsy instruments, laser ablation fibers, and/or other surgical, diagnostic, or therapeutic tools. The medical instrument 726 may be used with an imaging instrument (e.g., an image capture probe) within the flexible body 716. The imaging instrument may include a cable coupled to the camera for transmitting the captured image data. In some embodiments, the imaging instrument may be a fiber-optic bundle, such as a fiberscope, that couples to an image processing system 731. The imaging instrument may be single or multi-spectral, for example capturing image data in one or more of the visible, infrared, and/or ultraviolet spectrums. The medical instrument 726 may be advanced from the opening of channel 721 to perform the procedure and then be retracted back into the channel 721 when the procedure is complete. The medical instrument 726 may be removed from the proximal end 717 of the flexible body 716 or from another optional instrument port (not shown) along the flexible body 716.

The flexible body 716 may also house cables, linkages, or other steering controls (not shown) that extend between the drive unit 704 and the distal end 718 to controllably bend the distal end 718 as shown, for example, by broken dashed line depictions 719 of the distal end 718. In some embodiments, at least four cables are used to provide independent “up-down” steering to control a pitch of the distal end 718 and “left-right” steering to control a yaw of the distal end 718. Steerable elongate flexible devices are described in detail in U.S. Pat. No. 9,452,276, filed Oct. 14, 2011, disclosing “Catheter with Removable Vision Probe,” and which is incorporated by reference herein in its entirety. In various embodiments, medical instrument 726 may be coupled to drive unit 704 or a separate second drive unit (not shown) and be controllably or robotically bendable using steering controls.

The information from the tracking system 730 may be sent to a navigation system 732 where it is combined with information from the image processing system 731 and/or the preoperatively obtained models to provide the operator with real-time position information. In some embodiments, the real-time position information may be displayed on the display system 610 of FIG. 6 for use in the control of the medical instrument system 700. In some embodiments, the control system 612 of FIG. 6 may utilize the position information as feedback for positioning the medical instrument system 700. Various systems for using fiber optic sensors to register and display a surgical instrument with surgical images are provided in U.S. Pat. No. 8,900,131, filed May 13, 2011, disclosing “Medical System Providing Dynamic Registration of a Model of an Anatomic Structure for Image-Guided Surgery,” which is incorporated by reference herein in its entirety.

In some embodiments, the medical instrument system 700 may be teleoperated within the medical system 600 of FIG. 6. In some embodiments, the manipulator assembly 602 of FIG. 6 may be replaced by direct operator control. In some embodiments, the direct operator control may include various handles and operator interfaces for hand-held operation of the instrument.

The systems and methods described herein can be provided in the form of tangible and non-transitory machine-readable medium or media (such as a hard disk drive, hardware memory, optical medium, semiconductor medium, magnetic medium, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. Programmed instructions may be implemented as a number of separate programs or subroutines, or they may be integrated into a number of other aspects of the systems described herein. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. In one embodiment, the control system supports wireless communication protocols such as Bluetooth, IrDA, HomeRF, IEEE 802.11, DECT, and Wireless Telemetry. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

Medical tools that may be delivered through the elongate flexible devices or catheters disclosed herein may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. Medical tools may include image capture probes that include a stereoscopic or monoscopic camera for capturing images (including video images). Medical tools may additionally house cables, linkages, or other actuation controls (not shown) that extend between their proximal and distal ends to controllably bend the distal ends of the tools. Steerable instruments are described in detail in U.S. Pat. No. 7,316,681, filed Oct. 4, 2005, disclosing “Articulated Surgical Instrument for Performing Minimally Invasive Surgery with Enhanced Dexterity and Sensitivity” and U.S. Pat. No. 9,259,274, filed Sep. 30, 2008, disclosing “Passive Preload and Capstan Drive for Surgical Instruments,” which are incorporated by reference herein in their entireties.

The systems described herein may be suited for navigation and treatment of anatomic tissues, via natural or surgically created connected passageways, in any of a variety of anatomic systems, including the lung, colon, stomach, intestines, kidneys and kidney calices, bladder, liver, gall bladder, pancreas, spleen, ureter, ovaries, uterus, brain, the circulatory system including the heart, vasculature, and/or the like.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art. The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

Various aspects of the subject matter described herein are set forth in the following numbered examples.

Example 1. A method for performing a medical procedure comprises: surveying an anatomic cavity of a patient using an elongate flexible device, the surveying including: receiving commands for navigating the elongate flexible device within an interior space of the anatomic cavity and saving first location data from a first localization sensor coupled to the elongate flexible device. The method also comprises generating a three-dimensional (3D) anatomic model based, at least in part, on the first location data; receiving first image data from an imaging device coupled to the elongate flexible device; receiving second location data from the first localization sensor when an object within the anatomic cavity is visible in the first image data; updating the 3D anatomic model to include the object based, at least in part, on the second location data; and providing guidance for deploying an access tool from an external location to the object in the 3D anatomic model.

Example 2. The method of example 1 further comprises receiving second image data from the imaging device, wherein the object is less visible within the second image data than the first image data; receiving a command to reposition the elongate flexible device to a second position relative to the object within the second image data; and receiving third location data from the localization sensor when the object is visible in the second image data.

Example 3. The method of example 2, further comprises updating a location of the object in the 3D anatomic model based, at least in part, on the third location data.

Example 4. The method of example 3, further comprises determining a distance between the object and a distal end portion of the elongate flexible device, wherein updating the location of the object in the 3D anatomic model is further based on the distance.

Example 5. The method of any one of examples 2-4, further comprises determining one or more access paths for deploying the access tool from the external location to the object based, at least in part, on the 3D anatomic model.

Example 6. The method of example 5 wherein each of the one or more access paths includes an insertion position and an insertion angle for the access tool.

Example 7. The method of any of examples 2-4, further comprises updating the 3D anatomic model to include at least one sensitive tissue structure.

Example 8. The method of example 7 wherein the 3D anatomic model is updated to include the at least one sensitive tissue structure based, at least in part, on general anatomic information.

Example 9. The method of example 8, further comprises: receiving external imaging data; registering the external imaging data to the 3D anatomic model; and updating a position of the at least one sensitive tissue structure in the 3D anatomic model based on the external imaging data.

Example 10. The method of any one of examples 7-9 wherein the one or more access paths is configured to avoid the at least one sensitive tissue structure.

Example 11. The method of example 10, further comprises characterizing the one or more access paths based on at least one of path length, proximity to sensitive anatomy, or anatomical approach.

Example 12. The method of example 11, further comprises receiving access location data including a current position and a current angle of the access tool.

Example 13. The method of example 12, further comprising updating the one or more access paths based on the access location data.

Example 14. The method of example 12 or example 13, further comprising registering the first localization sensor to a second localization sensor, wherein the second localization sensor is coupled to the access device.

Example 15. The method of example 1 wherein the anatomic cavity includes an inner cavity of a kidney, and the object includes a kidney stone.

Example 16. A non-transitory, computer-readable medium storing instructions thereon that, when executed by one or more processors of a computing system, cause the computing system to perform the method of any one of example 1-15.

1. A system for performing a medical procedure, the system comprising:
an elongate flexible device including a distal end portion;
an imaging device coupled to the distal end portion of the elongate flexible device, wherein the imaging device is configured to obtain at least one image viewed from the distal end portion of the elongate flexible device;
a sensor system including at least one first sensor carried by the elongate flexible device, wherein the at least one first sensor is configured to obtain location data of the elongate flexible device;
a processor operably coupled to the elongate flexible device and the sensor system; and
a memory operably coupled to the processor, the memory storing instructions that, when executed by the processor, cause the system to perform operations comprising:
generating a three-dimensional (3D) anatomic model including an object within an anatomic cavity and one or more sensitive tissue structures near the anatomic cavity; and
providing guidance for deploying an access tool along at least one percutaneous access path through a patient's skin to the object based, at least in part, on the 3D anatomic model.

2. The system of claim 1 wherein the one or more sensitive tissue structures are outside of the anatomic cavity.

3. The system of claim 1 wherein the operations further comprise estimating a location of the one or more sensitive tissue structures relative to the anatomic cavity based, at least in part, on preoperative data.

4. The system of claim 3 wherein the preoperative data includes one or more patient characteristics.

5. The system of claim 1 wherein the operations further comprise determining a location of the one or more sensitive tissue structures relative to the anatomic cavity based, at least in part, on intraoperative data.

6. (canceled)

7. The system of claim 1, wherein the operations further comprise:
receiving external imaging data;
registering the external imaging data to the 3D anatomic model; and
estimating a location of the one or more sensitive tissue structures relative to the anatomic cavity based, at least in part the external imaging data.

8. The system of claim 1 wherein the operations further comprise estimating a location of the one or more sensitive tissue structures relative to the anatomic cavity based, at least in part, on general anatomic information.

9. The system of claim 1, wherein the 3D anatomic model is based, at least in part, on first location data from the at least one first sensor as the elongate flexible device is navigated within an interior space of an anatomic cavity and the object is based on second location data from the at least one first sensor when the object is within the at least one image.

10. The system of claim 1 wherein the at least one first sensor is configured to obtain location data of an interior space of an anatomic structure, wherein the location data includes point cloud data, and the 3D anatomic model is generated based, at least in part, on the point cloud data.

11. The system of claim 1 wherein the operations further comprise:
identifying a location of the object within the anatomic cavity based, at least in part, on the image of the object and on the location data obtained by the at least one first sensor.

12. The system of claim 11 wherein identifying the location comprises determining a distance between the object and a distal end portion of the elongate flexible device.

13. The system of claim 1 wherein the operations further comprise updating the 3D anatomic model.

14. The system of claim 13 wherein updating the 3D anatomic model comprises updating a location of the object based, at least in part, on the location data from the at least one first sensor.

15. The system of claim 14, wherein the at least one image includes a first image captured at a first position of the elongate flexible device and a second image captured at a second position of the elongate flexible device, wherein the object is less visible within the first image than the second image.

16-17. (canceled)

18. The system of claim 1 wherein the sensor system includes at least one of a shape sensor or an electromagnetic (EM) sensor.

19. The system of claim 1 wherein the percutaneous access path is different from an endoluminal access path for introducing the elongate flexible device into the anatomic cavity.

20. The system of claim 19 wherein the at least one percutaneous access path is a linear or curved path, and wherein the at least one percutaneous access path is chosen to avoid the one or more sensitive tissue structures.

21. (canceled)

22. The system of claim 1 wherein the sensor system further includes at least one second sensor carried by the access tool.

23. The system of claim 22, wherein the operations further comprise registering the at least one second sensor to the at least one first sensor.

24. The system of claim 23 wherein the guidance includes:
a target position and a target angle for the access tool to create the percutaneous access path, and
a current position and a current angle of the access tool obtained from the at least one second sensor.

25-28. (canceled)
